(19) United States
(12) Patent Application Publication
(10) Pub. No.: US 2010/0112675 A1
(43) Pub. Date: May 6, 2010

(54) MULTI-STAGED COMPOSTING

(75) Inventor: Bryan E. Imber, Victoria (CA)

Correspondence Address: KNOBBE MARTENS OLSON & BEAR LLP, 2040 MAIN STREET, FOURTEENTH FLOOR, IRVINE, CA, 92614, US

(73) Assignee: ZInternational Composting Corporation, Victoria, BC (CA)

(21) Appl. No.: 11/912,132

(22) PCT Filed: Apr. 20, 2006
(86) PCT No.: PCT/CA06/00636

§ 371 Date: Nov. 12, 2009

Related U.S. Application Data
(60) Provisional application No. 60/672,994, filed on Apr. 20, 2005.

(51) Int. Cl. C02F 3/34; C12M 1/00

(52) U.S. Cl. 435/262; 435/290.4

(57) ABSTRACT

In various embodiments, the invention provides staged processes, or systems, for composting organic materials, such as waste plant and animal matter. In one aspect, the invention involves the staged passage of organic material through alternative composting ecologies, to optimize the diversity of decay organisms that may work on the organic materials. In some embodiments, a robust, well mixed thermophilic environment is created for primary stage aerobic composting. The primary compost produced by this stage may be transferred to an alternative stratified composting ecology, in which the secondary compost descends over time from a relatively stable layer of residual thermophilic aerobic composting to underlying layers that involve non-thermophilic aerobic degradation of the organic material.

### FIELD

The present invention relates to composting of organic material. More specifically, the invention provides systems and processes that may be adapted for continuous composting that passes an organic material through thermophilic and non-thermophilic stages, thereby supporting a diversity of composting ecologies.

### BACKGROUND

Compost may be broadly defined as partly decayed organic material. In biochemical terms, the process of composting generally involves the microbiological degradation of organic compounds by metabolic processes. Composting is typically carried out so that the final product is well suited for application to soils as a fertilizer, to increase the humus content of soil (the brown or black organic fraction of the soil that consists of partially or wholly decayed vegetable or animal matter).

In many jurisdictions, there are regulations that govern the composition o f materials such as compost that are to be discharged into the environment. For example, Regulation (EC) No 1774/2002 of the European P arliament and of the Council sets out regulations for animal by-products that are not intended for human consumption, which may for example include composts. Such regulations typically relate in part to need to ensure that composts are appropriately treated to kill or inactivate pathogenic organisms that may be found in the raw organic material that is treated. For example, Regulation (EC) No 1774/2002 dictates that at least some materials used as raw material in a composting plant must be submitted to the following minimum treatment requirements: (a) maximum particle size before entering the composting reactor: 12 mm; (b) minimum temperature in all material in the reactor: 70° C.; and (c) minimum time in the reactor at 70° C. (all material): 60 minutes. These regulations also proscribe standards relating to the presence of some potentially pathogenic organisms in samples of the digestion residues or compost, such as: Salmonella: absence in 25 g: n=5, c=0, m=0, M=0; Enterobacteriaceae: n=5, c=2, m=10, M=300 in 1 g; where: n=number of samples to be tested; m=threshold value for the number of bacteria; the result is considered satisfactory if the number of bacteria in all samples does not exceed m; M=maximum value for the number of bacteria; the result is considered unsatisfactory if the number of bacteria in one or more samples is M or more; and c=number of samples the bacterial count of which may be between m and M. There is accordingly a need to provide composting systems that are capable of meeting various regulatory requirements relating to the treatment and composition of composts.

### SUMMARY

In various embodiments, the invention provides staged processes, or systems, for composting organic materials, such as waste plant and animal matter. In one aspect, the invention involves the passage of organic material through alternative composting ecologies, to optimize the diversity of decay organisms that may work on the organic materials. In some embodiments, a robust, well mixed thermophilic environment is created for primary stage composting. The primary compost produced by this stage may be transferred to an alternative stratified composting ecology, in which the secondary compost descends over time from a relatively stable layer of residual thermophilic composting to underlying layers that involve non-thermophilic degradation of the organic material. In the second stage, the absence of vigorous mixing allows alternative composting ecologies to establish themselves in a vertical gradient. In this way, the vigorous mixing and thermophilic degradation of the organic material in the primary stage, conditions the compost for subsequent incubation in the alternative stratified ecologies of the second stage. It has been found that this combined approach is advantageous in reducing the viability of pathogenic organisms that may be present in the raw organic feedstock.

In exemplary embodiments, the processes and systems of the invention may involve introducing raw organic materials into a primary composter, which may be an aerobic composter. The raw organic material may for example include viable pathogenic organisms, such as microorganisms that at certain concentrations or in certain circumstances are capable of producing or exacerbating plant or animal diseases. Once introduced, the raw organic material provides a primary composting mixture within the primary composter. This mixture may be displaced in the primary composter, for example longitudinally displaced, so that the primary composting mixture moves from the input end of the primary composter to an output end of the composter.

The transit of the primary composting mixture through the primary composter may take place over an interval defined as the primary composting time. During this period of time, the primary composting mixture may be mixed in the primary composter, so that vertical layers of the primary composting mixture are intermixed during the primary composting time, in the primary composter. Primary composting conditions may be sustained in the primary composter so that thermophilic organisms aerobically degrade the organic material. For example, a primary thermophilic composting temperature of at least 70 C. may be maintained in at least a portion of the primary composter for a primary thermophilic composting time. The duration of this thermophilic treatment may for example be at least one hour, to produce a primary compost;

The primary compost may be transferred from the output end of the primary composter to a secondary composter, which may be an aerobic composter, to provide a secondary composting mixture within the secondary composter. The secondary composting mixture may be vertically displaced in the secondary composter, so that the secondary composting mixture descends from an upper layer in the secondary composter to a lower layer in the secondary composter during a secondary composting time. In this way, the secondary composter is operable to provide a vertically stratified secondary composting mixture.

Composting conditions in the secondary composter may be monitored and maintained so that the upper layer of vertically stratified secondary composting mixture supports continued degradation of the organic material by thermophilic organisms. The conditions and timing of the primary composting stage may accordingly be modulated so that the primary compost is sufficiently biologically active to support further thermophilic degradation in the secondary composter. For example, secondary thermophilic composting may take place at a temperature of at least 70 C. for a duration defined as the secondary thermophilic composting time, which may for example be at least one hour.

Conditions may be sustained in the secondary composter so that a lower layer of the vertically stratified secondary composting mixture supports non-thermophilic degradation of the organic material by non-thermophilic organisms, which may be aerobic or anaerobic or both. For example, non-thermophilic composting may take place at a secondary thermophilic composting temperature below 70 C. for a secondary thermophilic composting time, such as at least one hour, to produce a secondary compost. The entire composting process of the invention may for example be carried out so as to reduce the viability of pathogenic organisms in the raw organic material.

In some embodiments, processes of the invention may be continuous, in the sense that material moves continuously through the composting systems and stages of the invention. In this way, for example, the robust thermophilic composting environment created in the primary composter may be transferred to the initial stage of the secondary composter, so that a second stage of thermophilic composting can take place. This requires an appropriate modulation of the primary composting process conditions so as to provide a primary compost that will support continued thermophilic composting in the secondary composter. Similarly, the continuous removal of composted material from the secondary composter may be modulated so as to facilitate a gradual ecological shift in the compost as it descends the secondary composter, from a thermophilic composting ecology to a non-thermophilic ecology, with opportunities within this ecological shift for a wide variety of organisms to degrade the organic material.

### DETAILED DESCRIPTION

The invention provides apparatuses and processes for composting. In one aspect, the processes of the invention may be used in the treatment of organic waste, such as plant or animal by-products, municipal wastes, or other compositions containing raw organic material suitable for composting. The raw organic waste material or feed material may be treated to provide a compost or fertilizer suited for general use, for example by virtue of the absence of potentially pathogenic or ecologically disadvantageous organisms. In some embodiments, the raw organic feed material may be reduced in size and mixed prior to being introduced into a composting apparatus or “composter”.

In various aspects, the processes of the invention utilize relatively high composting temperatures, such as temperatures in excess of a threshold value at which thermophilic organisms are active in biological degradation of organic materials, which may be defined as “thermophilic composting temperatures”. These threshold thermophilic temperatures may also be selected and maintained so as to inactivate undesirable organisms in the raw organic feedstock. For example, thermophilic temperatures may be in excess of 70° C., or in excess of a threshold value which is any integer or decimal value between 40° C. and 80° C., such as 70° C., 71° C., 72° C., 73° C., 74° C., 75° C. or 80° C. These thermophilic composting temperatures may be modulated at various stages of the processes of the invention, so that alternative temperatures are used which are suitable for the growth of alternative thermogenic or thermophilic organisms. Alternatively, a single thermophilic temperature, or temperature range may be imposed throughout the process.

“Thermophilic organisms” as used herein refers to any heat-tolerant organism; such as bacteria, yeast, or fungi regardless of whether the heat tolerance is a necessity for metabolism and growth, or merely a capability. For example, the term encompasses organisms metabolically active above threshold temperatures from 40° C. to 80° C.

“Pathogenic organism” as used herein includes any organism capable of causing, producing or sustaining any disease or adverse effect in animals, including humans, as well as in plants. Examples include the fecal coliforms, Escherischia coli, Salmonella spp, fecal streptococci, or other fecal contaminants, spore-forming Bacillus spp, anaerobic Clostridia, or viruses. In addition, pathogenic organisms as used herein includes organisms that are ecologically pathogenic, in the sense that they disrupt or introduce ecological changes that are not desirable, for example leading to a decrease in the usefulness of a compost for use as a fertilizer.

To achieve desired composting conditions, processes of the invention may involve controlling movement and mixing of the composting material based on measurements of the status of the compost, such as measurements of the temperature and/or oxygen concentration in the composting material. In this way, one or more suitable or optimal environment(s) for thermogenic or thermophilic organisms may be achieved. The composting organisms, including thermogenic or thermophilic organisms, may be endogenous to the raw organic material used as feedstock, or the organisms may be provided as an inoculum or seed during processes of the invention. The use of a variety of organisms and materials in a composting inoculum is for example discussed in WO 2004/035508, which is hereby incorporated by reference.

The temperature at various stages in the processes of the invention may be monitored and controlled by a wide variety of mechanisms. For example, thermogenic organisms in the treated material may be cultured so as to produce high temperatures, such as temperatures in excess of a threshold value, such as a value of about 70° C. or an alternative threshold as set out above. Alternatively, or in addition, a composting apparatus may be heated and or insulated to provide means for sustaining a desired temperature, such as any of the threshold temperature set out above. By maintaining or establishing a suitably high temperature, growth of thermophilic organisms in the compost is encouraged. Once suitable temperatures are achieved, and colonies of thermophilic organisms are established, further heating may not be required. The temperature may be elevated above a selected threshold temperature, which may be defined as a thermophilic composting temperature, for a selected length of time, such as from one to four days, or any number of hours from 1 to 100, such as 1, 2, 24, 48 or 96 hours.

Measurements may be made of the temperature and oxygen content in the composting material, or of other parameters of interest. Such measurements may form part of a control system for adjusting composting conditions so as to be suitable for growth of thermophilic bacteria, or to determine the speed and efficacy of the composting process. For example, analytical data such as metal content, and salmonella and coliform counts may be determined from time to time, for example on a daily basis. The measurements may be taken on a continuous or discontinuous basis, and the frequency of analysis of the data may be adjusted as part of the control systems of the invention. For example, if oxygen levels are determined to be low, air may be injected into a composter, such as a rotational vessel used as a primary composter, to provide more oxygen for thermophilic bacteria. Exhaust gases may be collected and treated, for example for odor and biological oxygen demand. The air and water (steam) components of exhaust gases may be separated, and the water may be treated in a water treatment system while the air may be passed through a biofilter.

In some embodiments, the invention provides processes that link two composting stages, a primary and secondary stage, each of which includes composting environments at relatively high temperatures, such as temperatures in excess of 70° C. or any threshold temperature set out above. The first or primary stage may for example be carried out in a composting apparatus that includes a rotational vessel (i.e., a vessel capable of rotating). In rotational primary composters, the rotational speed can be controlled via inputs to a programmable logic controller (PLC).

In some embodiments, a secondary composting stage may be carried out in a composting apparatus that includes a stacking system, such as a vertical bin. In some embodiments, the rotational vessel and the stacking system may be operably linked to be part of a unitary composting apparatus, and the transfer of material from the primary rotational composting vessel to the secondary stacking composter may be automated. Accordingly, in some embodiments, the composting system of the invention includes a primary composter that is a rotational vessel, followed by a secondary composter that is a compost stacking composter.

The rotational vessel or composter may for example be of any shape that allows for rotation, such as cylindrical, and may be made of steel or any other suitable material. Rotary composters and control systems therefor are for example disclosed in U.S. Pat. Nos. 6,001,641 and 6,110,733, which are hereby incorporated by reference. In selected embodiments, the dimensions of a cylindrical rotary composter may for example be at least about 10 ft in diameter and at least about 50 ft in length. Larger diameters or longer lengths may be used to increase capacity. A wide variety of rotational speeds may be used, such as speeds of from 1 to 5 rev/hour, and rotation may be modulated within a range, in response to conditions with the composter, or maintained at about a selected value, such as 1, 2, 3, 4 or 5 rev/hour.

The primary composter, such as a rotational vessel, may be positioned at an incline, to facilitate the flow of composting material from the input end to the output end of the composter. The primary composter may also be configured to cause the compost to tumble or mix during rotation, for example by positioning angular bars or plates on the inside of the rotational vessel. Due to the incline of the rotational vessel and the internal configuration, e.g., plate arrangement, material can move down the rotational vessel by gravity as the rotational vessel is turned. Waste or feed material may be fed into the elevated end of the composter via input hatches by any suitable method, e.g., by conveyer. The location of the hatches may vary, for example, the hatches may be located at the top, center, or at one end of the composter vessel. The hatches may be configured so as to enable filling of the vessel while the vessel is at rest, or while it is rotating.

The material may be retained in the rotational vessel for a suitable length of time, e.g., at least 72 hours. One or more instrumentation package(s) may be installed for providing continuous oxygen and temperature measurements, or for monitoring any other parameter of interest. In some embodiments, oxygen sensing packages may be installed in at least six locations and a manifold can be made from a suitable material such as stainless steel so that oxygen from the six locations may be sampled sequentially, or if desired, substantially simultaneously. The instrumentation packages may be located at the edge of the rotational vessel and/or within the rotational vessel. In some embodiments, the determination of oxygen may require that the oxygen sensor be mounted in a manner that it does not rotate.

Measurements of temperature and oxygen may be taken on a continuous basis and used for example to control the rotational speed of the rotational vessel. Oxygen and temperature controls may be used to provide the correct rotational speeds to promote thermophilic bacteria. A feedback loop connecting the rotation of the rotational vessel to the detected oxygen concentrations in the rotational vessel may be used. The rotational vessel may be operated with oxygen concentrations less than 10 mg/L and more than 2 mg/L. If the oxygen levels fall below 2 mg/L, the speed of rotation may be automatically increased. Air may be drawn from the rotational vessel to the sensor. Exhaust gases may be collected and treated for odour and biological oxygen demand from the first heating stage.

Waste or feed material transformed into first stage compost may exit the cylindrical vessel by way of suitably dimensioned discharge hatches. The hatches may be operated by any suitable mechanisms, e.g., electronic motors and actuator mechanisms t hat allow the hatches to be opened automatically via the PLC. In some embodiments, the exiting first stage compost may be contained in a suitable conveyer (e.g., a screw conveyer fitted with electronic gates) and sent to a stacking system (e.g., vertical bin).

In a stacking system, the hot compost on the top may cause a chimney effect and draw air up through the compost through air vents located at the bottom of the container or bin. Generally, a temperature profile develops in the bin such that temperatures in excess of 700 C. are present at a few feet depth. Temperatures of 40-500 C. are generally present at 10 to 15 feet depth and exit temperatures are usually less than 300 C. Generally, thermophilic organisms at the top cause temperatures of 70-800 C. to be reached in the top few feet. In one embodiment, material moves down the stacking system container as material is removed from the bottom. New material is added to the top and processed compost moves down the stacking system and is considered finish compost upon exiting the stacking system.

The stacking system (e.g., vertical bin) may be suitably dimensioned and may include one or more input conveyers at the top and one or more exit conveyers on the bottom. The first stage compost material is generally spread out evenly on the top surface of the bin. The stacking system may be insulated to, for example, provide suitable or optimised growing conditions for thermophilic bacterial processing of the second stage compost.

The sides of the stacking system may be solid, lined with plastic or any other suitable material to reduce corrosion and may be insulated. In another embodiment, the stacking system may be adapted so that the system does not need to be sheltered. This can be achieved for example by providing roofs made of a suitable material e.g., plastic, for the stacking system.

The stacking system may include instrumentation able to control the oxygen concentrations in the bin to for example provide thermophilic bacterial growth. Typically oxygen is maintained above 2 mg/L. The natural air flow created by the chimney may be sufficient. However, air fan systems may also be used. For example, if the height of the stacking system is increased, an air fan system may augment or supplant natural air flow. Data from for example oxygen sensors may be used to turn on and off air fans that force air in at the base of the stacking system. The stacking system may include aeration tubes for both passive and active aeration processes.

The stacking system may include a permeable membrane able to retain odour. In some embodiments, a semi-permeable membrane is used to cover the top surface of the bin. This membrane allows air to escape but does not allow the larger organic acid molecules to pass.

In some embodiments, both the rotational vessel and the stacking system may be insulated and weather proofed so as to be used in any environment. In some embodiments, both the rotational vessel and the stacking system may include heating capability (e.g., by insulation or fitted heaters) to help maintain elevated temperatures and to ensure thermophilic organism populations can be maintained.

In some embodiments, both the rotational vessel and the stacking system may be controlled by a PLC allowing direct and/or remote operation. In some embodiments, both the rotational vessel and the stacking system may include instrumentation able to provide continuous oxygen and temperature measurements, or for monitoring any other parameter of interest. In some embodiments, material from a specific input day can be identified and the analytical data connected to this material can be attached thereto.

Thus, the invention provides in part for processing of organic matter or other feed material to produce compost and fertilizer products. In some embodiments, the processes and apparatuses of the invention are able to produce compost and fertilizer products in a time and energy efficient manner. In some embodiments, the two stage process according to the invention may allow for rapid composting. In some embodiments, pathogen reduction in the two stage process according to the invention may allow for a reduced risk of pathogen survival in the final product. In some embodiments, the ability for additional analyses may increase the quality of the final product. In some embodiments, the processes and apparatuses of the invention are cost effective and safe due to for example the lack of requirement for human intervention during normal processing, which may reduce contamination of the final product.

The following examples are provide to illustrate selected embodiments of the invention do not limit the scope of the invention.

### Examples

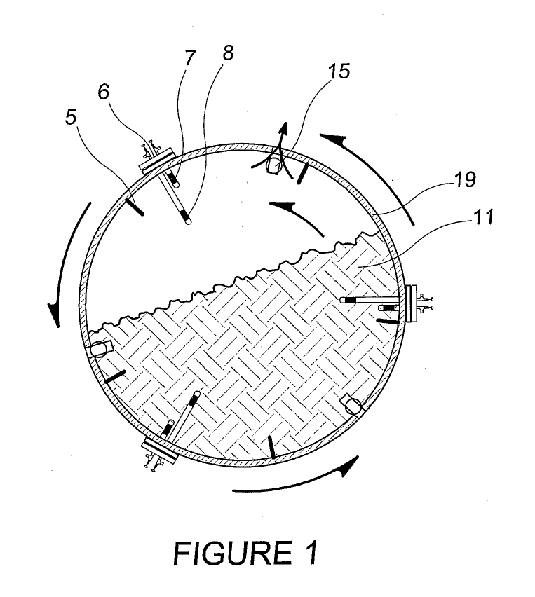

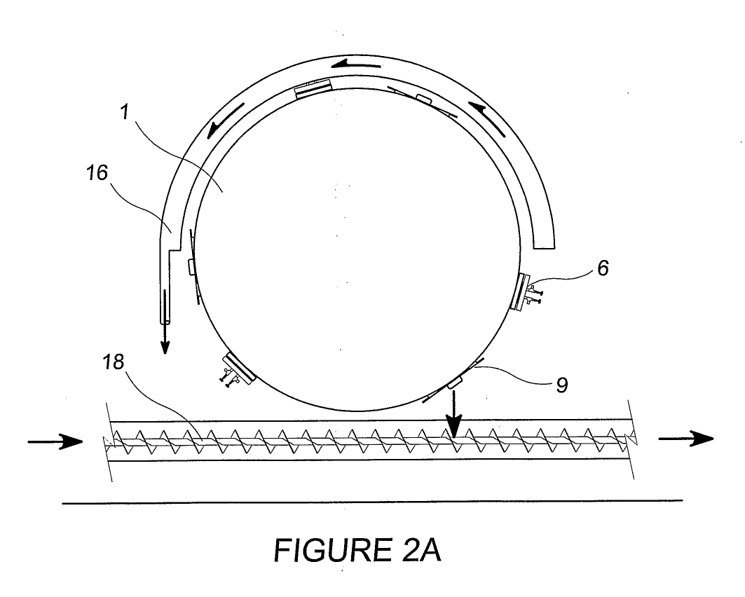

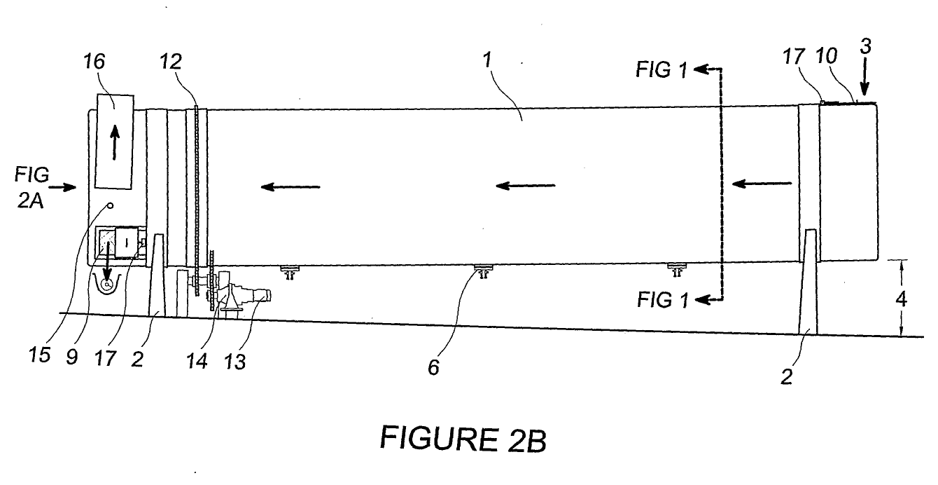

FIG. 1 shows a cross section of an embodiment of a component of the composting system, a cylindrical vessel.

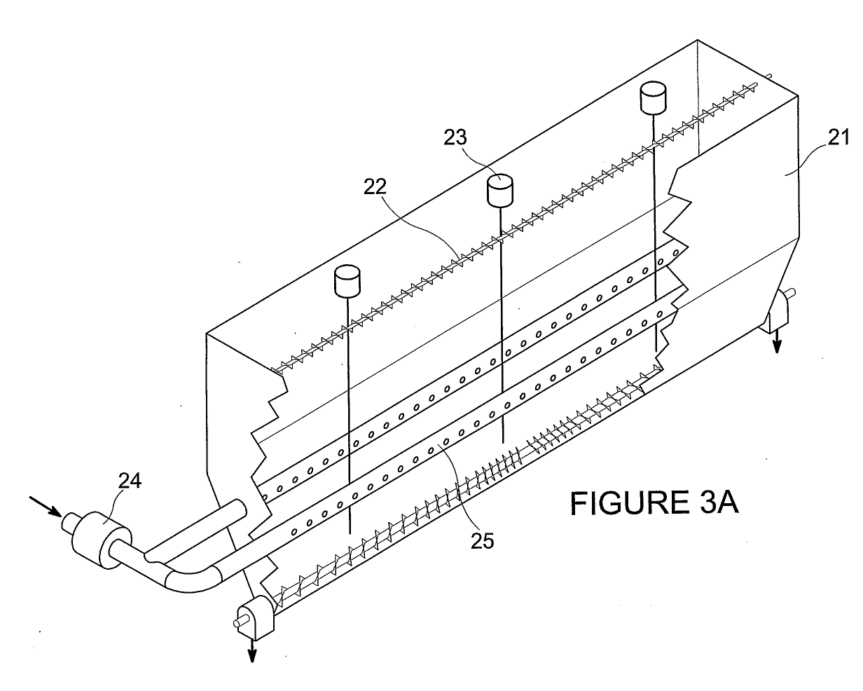

The dimensions of the cylinder are at least about 10 ft in diameter and about 50 ft in length (e.g., about 55 ft). The cylinder is held or seated on two saddles 2 and is rotated about its longitudinal axis (see FIGS. 1 and 2B). The saddles 2 use a friction reduced plastic contact, such as UHMU, although any other suitable material may be used.

The cylindrical vessel is driven by a five horsepower motor 13 connected to a 920:1 gear box 14 (FIG. 2B). The motor is fitted with a brake to ensure that the loading hatch is correctly positioned and is slanting during loading. The cylindrical vessel is connected to a chain drive mechanism 12 that surrounds the vessel (FIG. 2B), allowing for positive traction. This may provide added stability in the event for example of an earthquake.

The cylindrical vessel is positioned at an incline, to allow material to flow from the input end to the output end (FIG. 2B). The cylindrical vessel has angular bars or internal plate arrangement(s) 5 on its inside (FIG. 1) to allow the compost to tumble. Due to the incline of the vessel and the internal plate arrangement 5, material moves down the vessel by gravity as the cylinder is turned. Waste or feed material is fed by conveyer into the elevated end of the composter via input hatches.

Instrumentation package(s) 6 are installed (FIG. 1) for providing continuous oxygen and temperature measurements, or for monitoring any other parameter of interest. An exemplary arrangement for the oxygen and temperature sensors is shown. Exhaust gases are collected and treated for odour and biological oxygen demand from the first heating stage in the cylindrical vessel. Steam may emanate from the cylindrical vessel by way of three or more large (e.g., 8 ft) ball valves 9 that open under gravity when in the top of the turn (FIG. 2A). Gas is collected using a suction collecting device 16 (FIG. 2B). FIGS. 2A-B show details of the odor control systems 16. The air and water components are separated, and the water is treated in a water treatment system while the air is passed through a biofilter.

Compost exits the cylindrical vessel by way of three discharge hatches 17 dimensioned 4″ by 22″ (FIG. 2B). The hatches are operated by electronic motors and actuator mechanisms that allow the hatches to be opened automatically via the PLC. An exit screw conveyer is situated under the hatches and is fitted with a plastic shield (or any other suitable material) to ensure all the exiting compost is contained in the screw conveyer. The exit material is then elevated to 25 ft using chain or bucket conveyers. In one embodiment, material exiting the cylindrical vessel may be sent to a stacking system.

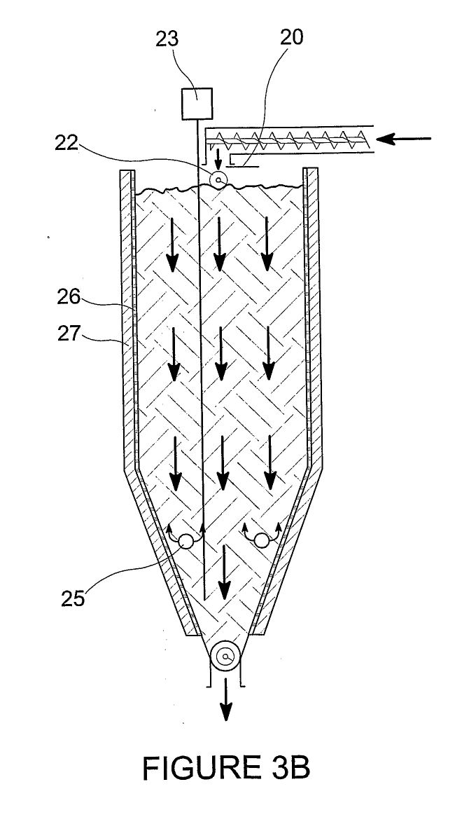

In an alternate embodiment, the composter or composting system may include a stacking (e.g., vertical bin) system, including a vessel about 20 ft in height, about 45 ft in length, and about 8 ft in width, with one or more input conveyer(s) 22 on the top (FIG. 3B) and one or more exit conveyer(s) on the bottom (FIG. 3A). The hot compost on the top cause a chimney effect and draws air up through the compost through air vents 35 located at the bottom of the container (FIG. 3B).

A screw conveyer fitted with electronic gates allows the first stage compost material to be placed in a vertical stacking bin. The top surface of compost material in the bin is spread out evenly by means of a leveling screw conveyer 22 (FIG. 3B). Three clusters of instruments 23 are placed in the vertical bin enabling profiles of temperature and oxygen to be determined (FIG. 3A).

### Other Embodiments

Although various embodiments of the invention are disclosed herein, many adaptations and modifications may be made within the spirit and scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the specification, the word “comprising” is used as an open-ended term, substantially equivalent to the phrase “including, but not limited to”, and the word “comprises” has a corresponding meaning. Citation of references herein shall not be construed as an admission that such references are prior art to the present invention. All publications are incorporated herein by reference as if each individual publication were specifically and individually indicated to be incorporated by reference herein and as though fully set forth herein. The invention includes all embodiments and variations substantially as hereinbefore described and with reference to the examples and drawings.

1. A staged process for composting an organic material, the process comprising:
introducing a raw organic material into a primary composter, the raw organic material comprising viable pathogenic organisms, to provide a primary composting mixture within the primary composter;
longitudinally displacing the primary composting mixture in the primary composter, so that the primary composting mixture moves from an input end of the primary composter to an output end of the primary composter during a primary composting time;
mixing the primary composting mixture in the primary composter, so that vertically spaced apart layers of the primary composting mixture are intermixed during the primary composting time in the primary composter;
sustaining primary composting conditions in the primary composter so that thermophilic organisms degrade the organic material, at a primary thermophilic composting temperature of at least 70° C. for a primary thermophilic composting time of at least one hour, to produce a primary compost;
transferring the primary compost from the output end of the primary composter to a secondary composter, to provide a secondary composting mixture within the secondary composter;
vertically displacing the secondary composting mixture in the secondary composter, so that the secondary composting mixture descends from an upper layer in the secondary composter to a lower layer in the secondary composter during a secondary composting time, to provide a vertically stratified secondary composting mixture;
sustaining upper secondary composting conditions in the secondary composter so that the upper layer of the vertically stratified secondary composting mixture supports continued degradation of the organic material by the thermophilic organisms in the secondary composter at a secondary thermophilic composting temperature of at least 70° C. for a secondary thermophilic composting time of at least one hour; and,
sustaining lower secondary composting conditions in the secondary composter so that the lower layer of the vertically stratified secondary composting mixture supports non-thermophilic degradation of the organic material by non-thermophilic organisms in the secondary composter at a secondary thermophilic composting temperature below 70° C. for a secondary thermophilic composting time of at least one hour, to produce a secondary compost in which the viability of the pathogenic organisms is reduced compared to the raw organic material.

2. A staged system for composting an organic material, the system comprising:
a primary composter having an input end for accepting a raw organic material, the raw organic material comprising viable pathogenic organisms, so that the primary composter contains a primary composting mixture;
means for longitudinally displacing the primary composting mixture in the primary composter, so that the primary composting mixture moves from the input end of the primary composter to an output end of the primary composter during a primary composting time;
means for mixing the primary composting mixture in the primary composter, so that vertically spaced apart layers of the primary composting mixture are intermixed during the primary composting time in the primary composter;
means for sustaining primary composting conditions in the primary composter so that thermophilic organisms degrade the organic material, at a primary thermophilic composting temperature of at least 70° C. for a primary thermophilic composting time of at least one hour, to produce a primary compost;
means for transferring the primary compost from the output end of the primary composter to a secondary composter, to provide a secondary composting mixture within the secondary composter;
means for vertically displacing the secondary composting mixture in the secondary composter, so that the secondary composting mixture descends from an upper layer in the secondary composter to a lower layer in the secondary composter during a secondary composting time, to provide a vertically stratified secondary composting mixture;
means for sustaining upper secondary composting conditions in the secondary composter so that the upper layer of the vertically stratified secondary composting mixture supports continued degradation of the organic material by the thermophilic organisms in the secondary composter at a secondary thermophilic composting temperature of at least 70° C. for a secondary thermophilic composting time of at least one hour; and,
means for sustaining lower secondary composting conditions in the secondary composter so that the lower layer of the vertically stratified secondary composting mixture supports non-thermophilic degradation of the organic material by non-thermophilic organisms in the secondary composter at a secondary thermophilic composting temperature below 70° C. for a secondary thermophilic composting time of at least one hour, to produce a secondary compost in which the viability of the pathogenic organisms is reduced compared to the raw organic material.

3. A staged system for composting an organic material, the system comprising:
a primary composter having an input end that accepts a raw organic material, the raw organic material comprising viable pathogenic organisms, so that the primary composter contains a primary composting mixture;
a conveyance mechanism that longitudinally displaces the primary composting mixture in the primary composter, so that the primary composting mixture moves from the input end of the primary composter to an output end of the primary composter during a primary composting time;
a mixing mechanism that mixes the primary composting mixture in the primary composter, so that vertically spaced apart layers of the primary composting mixture are intermixed during the primary composting time in the primary composter;
a primary compost monitoring and control system that sustains primary composting conditions in the primary composter so that thermophilic organisms aerobically degrade the organic material, at a primary thermophilic composting temperature of at least 70° C. for a primary thermophilic composting time of at least one hour, to produce a primary compost;
a primary compost transfer mechanism connecting the output end of the primary composter to a secondary composter, the transfer mechanism being operable to transfer the primary compost from the output end of the primary composter to the secondary composter, to provide a secondary composting mixture within the secondary composter;
the secondary composter being operable to vertically displace the secondary composting mixture, so that the secondary composting mixture descends from an upper layer in the secondary composter to a lower layer in the secondary composter during a secondary composting time, to provide a vertically stratified secondary composting mixture;
a secondary compost monitoring and control system that sustains upper secondary composting conditions in the secondary composter so that the upper layer of the vertically stratified secondary composting mixture supports continued degradation of the organic material by the thermophilic organisms in the secondary composter at a secondary thermophilic composting temperature of at least 70° C. for a secondary thermophilic composting time of at least one hour; and,
the secondary compost monitoring and control system sustains lower secondary composting conditions in the secondary composter so that the lower layer of the vertically stratified secondary composting mixture supports non-thermophilic degradation of the organic material by non-thermophilic organisms in the secondary composter at a secondary thermophilic composting temperature below 70° C. for a secondary thermophilic composting time of at least one hour, to produce a secondary compost in which the viability of the pathogenic organisms is reduced compared to the raw organic material.

4. The process of claim 1, wherein the composters are aerobic, and aerobic primary composting conditions are sustained in the primary composter so that thermophilic organisms aerobically degrade the organic material; and aerobic secondary composting conditions are sustained in the secondary composter so that the lower layer of the vertically stratified secondary composting mixture supports non-thermophilic aerobic degradation of the organic material by non-thermophilic organisms.

5. The process of claim 1, wherein composting is continuous in the primary and secondary composters, with the raw material being introduced into the primary composter while secondary compost is produced by the secondary composter.

6. The process of claim 5, wherein the primary composting conditions are monitored and adjusted to maintain selected primary composting process parameters.

7. The process of claim 5, wherein the upper or lower secondary composting conditions are monitored and adjusted to maintain selected secondary composting process parameters.

8. The process of claim 6, wherein the primary or secondary composting process parameters comprise temperature or oxygen content.

9. The process of claim 1, wherein the primary composter is an inclined rotational drum composter having a primary composter incline.

10. The process of claim 9, wherein the primary composter comprises an internal plate arrangement that operates in conjunction with the primary composter incline to longitudinally displace material along the primary composter as it rotates.

11. The process of claim 9, wherein the primary composting time is at least 48 hours.

12. The process of claim 9, wherein the drum composter is operated with oxygen concentrations less than about 10 mg/L and more than about 2 mg/L.

13. The process of claim 1, wherein upper and lower secondary composting conditions are sustained so that a temperature profile develops in the secondary composter such that upper secondary composting temperatures of at least 70° C. are sustained in an upper portion of the secondary composter, and middle secondary composting temperatures of about 40-50° C. are sustained in a middle portion of the secondary composter, and exit temperatures of the secondary compost are less than about 35° C.

14. The process of claim 1, wherein the temperature profile in the secondary composter draws air up through the secondary composter through air vents located in a lower region of the secondary composter.

15. The process of claim 1, wherein the concentration of oxygen in the secondary composter is maintained at above about 2 mg/L.

16. The process of claim 1, wherein the primary composter or the secondary composter are insulated and heated.
